(19) United States
(12) Patent Application Publication
(10) Pub. No.: US 2015/0044148 A1
(43) Pub. Date: Feb. 12, 2015

(54) Method for Treating Capillary Hemangiomas

(71) Applicant: Galderma Laboratories, Inc., Forth Worth, TX (US)

(72) Inventor: Warren J. Scherer, Lutz, FL (US)

(21) Appl. No.: 14/344,091

(22) PCT Filed: Oct. 15, 2012
(86) PCT No.: PCT/IB2012/002509

§ 371 Date: Mar. 11, 2014

Related U.S. Application Data
(60) Provisional application No. 61/548,838, filed on Oct. 19, 2011.

(51) Int. Cl. A61K 9/00; A61K 31/4174; A61N 5/10; A61K 38/21; A61B 18/20; A61K 31/498; A61K 45/06

(52) U.S. Cl. CPC A61K 9/0014; A61K 31/498; A61K 31/4174; A61K 45/06; A61K 38/212; A61B 18/20; A61N 5/10
USPC 424/59; 514/249; 514/401; 424/85.7; 606/2; 600/1

(57) ABSTRACT

The invention relates to a method of treating capillary hemangiomas in a human in need thereof by topically administering an effective amount of one or more alpha-2 adrenergic receptors agonist to the site of the capillary hemangiomas on the skin of the human.

### CROSS-REFERENCE TO RELATED APPLICATION

The present application claims benefit of U.S. provisional application Ser. No. 61/548,838, filed Oct. 19, 2011, which is incorporated herein by reference in its entirety.

### BACKGROUND OF THE INVENTION

Capillary hemangiomas, also known as, strawberry nevi, infantile hemangioma, juvenile hemangioma, hemangioblastoma, benign hemangioendothelioma, hypertrophic hemangioma are the most common facial, orbital and ocular adnexal tumors in children. It has been reported that as many as 10% of all children under 1 year of age have visible capillary hemangiomas. The incidence of these lesions is even higher, 23%, among premature infants less than 1000 gms. Capillary hemangiomas are benign, infiltrative neoplasms consisting of anastomosing vascular channels. They may present as small, isolated lesions or large disfiguring masses that may interfere with vision and/or induce amblyopia.

The natural history of capillary hemangiomas includes a series of biological phases. Initially, there is a proliferative phase with rapid tumor growth, usually beginning within the first 3-6 months after diagnosis. Following several months of growth, the lesions then stabilize and begin the process of involution. Most spontaneous involution will occur by age 5, however this process may continue until the end of the first decade.

There are several approaches to the treatment of capillary hemangiomas. Because of the high rate of spontaneous regression, observation alone is often sufficient. However, intervention is also often required, as in the case of large, amblyogenic, non-regressing or cosmetically unacceptable lesions. Aside from complete surgical excision, current medical treatments focus on methods that act to reduce blood flow by closing the vessels within the tumor via vasoconstriction, vascular embolization or coagulation. The most common treatment methods include systemic or intralesional corticosteroids, radiation, interferon and laser therapy.

Currently, intralesional injection of corticosteroid is the mainstay of treatment for capillary hemangiomas. With intralesional injection, a high, localized, dose of corticosteroid is achieved and many of the side effects of systemic steroids, such as adrenal suppression and growth retardation, can be avoided. Although successful, reported side effects of intralesional corticosteroid injection include accidental globe perforation, retrobulbar hemorrhage, central retinal artery occlusion, subcutaneous fat atrophy and yellow or white subcutaneous deposits.

Radiation therapy has also been shown to be effective in the treatment of capillary hemangioma. Unfortunately, radiation also carries substantial risks such as radiation retinopathy and radiation-induced oncogenesis. Currently, radiation treatment is reserved for situations in which there are no other effective therapies.

Interferon alpha-2a was initially developed as an antiviral agent but was subsequently found to have anti-angiogenic properties. Although the exact anti-angiogenic mechanism is unclear, it is thought to be due to an inhibition of vascular endothelial proliferation and subsequent vessel closure. Although encouraging results have been observed with interferon, it is associated with severe side effects such as leucopenia and neurotoxicity, e.g., spastic diplegia. As a result, this therapy is reserved for life or sight-threatening tumors that are resistant to corticosteroids.

Lasers have also been used to treat capillary hemangiomas. The various lasers employed include the carbon dioxide, argon, and neodymium YAG. The mechanism involves a direct vaso-obliterative and coagulative effect on the tumor vasculature. The side effects of laser treatment include thermo-destruction of tissue adjacent to the hemangioma, inability to penetrate past the laser-induced charring of the surface of the hemangioma and permanent scarring.

Due to the side effects of the current regimens used to treat capillary hemangiomas, a treatment method that safely results in a reduction of blood flow within the hemangioma, thereby closing the vascular elements within the tumor and subsequently resulting in growth reduction and induction of tumor involution, is needed.

### SUMMARY OF THE INVENTION

The invention relates to a method for treating capillary hemangiomas in a human in need thereof by topically administering a composition including an effective amount of one or more anti-capillary hemangioma active compounds or pharmaceutically acceptable salts thereof to the site of capillary hemangiomas on the skin of the human.

In one embodiment, the one or more anti-capillary hemangioma active compound or pharmaceutically acceptable salts thereof is xylometazoline, epinephrine, nerepinephrine, phenylephrine, methoxamine, guanabenz, guanfacine, alpha-methyl dopamine, amphetamine, methylphenidate, lofexidine, moxonidine, dexmedetomidine, mivazerol, brimonidine, oxymetazoline, or any combinations of such compounds or salts.

In another embodiment, the one or more anti-capillary hemangioma active compound or pharmaceutically acceptable salts thereof is xylometazoline, epinephrine, nerepinephrine, phenylephrine, methoxamine, or any combinations of such compounds or salts.

In yet another embodiment, the one or more anti-capillary hemangioma active compound or pharmaceutically acceptable salts thereof is guanabenz, guanfacine, alpha-methyl dopamine, amphetamine, methylphenidate, lofexidine, moxonidine, dexmedetomidine, mivazerol, or any combinations of such compounds or salts.

In still another embodiment, the one or more anti-capillary hemangioma active compound or pharmaceutically acceptable salt thereof is brimonidine, oxymetazoline, or combinations thereof.

Preferably, the pharmaceutically acceptable compound is brimonidine tartrate. The preferred amount of brimonidine tartrate present in the composition is a minimum of about 0.01% and a maximum of about 5%, based upon the total weight of the composition.

In another embodiment, the pharmaceutically acceptable salt is oxymetazoline hydrochloride. The preferred amount of oxymetazoline hydrochloriode present in the composition is a minimum of about 0.01% and a maximum of about 5%, based upon the total weight of the composition.

The composition may also include one or more additional pharmaceutically active ingredients. Some examples of additional pharmaceutically active compounds are selected from the group consisting of antibacterial agents, anthelmintic agents, antioxidant agents, steroidal anti-inflammatory agents, non-steroidal anti-inflammatory agents, antiangiogenic agents, and derivatives of retinoic acid. The composition may also include aloe, a sunscreen, a preservative, or combinations thereof.

In another embodiment, the only anti-capillary hemangioma active compound in the composition is xylometazoline, epinephrine, nerepinephrine, phenylephrine, methoxamine, guanabenz, guanfacine, alpha-methyl dopamine, amphetamine, methylphenidate, lofexidine, moxonidine, dexmedetomidine, mivazerol, brimonidine, oxymetazoline, a pharmaceutically acceptable salt thereof, or any combination of such compounds or salts. In yet another embodiment, the only pharmaceutically active ingredients for treating capillary hemangiomas are brimonidine or a pharmaceutically acceptable salt thereof or oxymetazoline or a pharmaceutically acceptable salt thereof; or a combination of brimonidine or a pharmaceutically acceptable salt thereof or oxymetazoline or a pharmaceutically acceptable salt thereof. In yet another embodiment, the only pharmaceutically active compounds of any kind are brimonidine or a pharmaceutically acceptable salt thereof or oxymetazoline or a pharmaceutically acceptable salt thereof, or a combination of brimonidine or a pharmaceutically acceptable salt thereof and oxymetazoline or a pharmaceutically acceptable salt thereof.

The composition may also be administered along with the systemic administration or intralesional injection of a corticosteroid. The composition may be administered along with another treatment for capillary hemangiomas such as radiation therapy, interferon therapy, or laser therapy.

### DETAILED DESCRIPTION OF THE INVENTION

The present invention relates to methods of treating capillary hemangiomas in a human patient in need thereof by topically administering a composition comprising an effective amount of one or more anti-capillary hemangioma active compounds, or pharmaceutically acceptable salts thereof, to the site of capillary hemangiomas on the skin of the patient. The anti-capillary hemangioma active compounds are alpha-2 adrenergic receptor agonists.

Capillary hemangioma, also known as strawberry nevus, strawberry hemangioma, infantile hemangioma, juvenile hemangioma, hemangioblastoma, benign hemangioendothelioma, and hypertrophic hemangioma, usually occur at birth or up to four weeks after birth. They are benign, infiltrative neoplasms consisting of anastomosing vascular channels that may present anywhere on the body as small, isolated lesions or large, disfiguring masses. The lesions are usually red or reddish-purple raised sores or massive, raised tumors with blood vessels. Capillary hemangiomas appear commonly on the head or neck areas of newborns.

Anti-capillary hemangioma active compounds or pharmaceutically acceptable salts thereof have now been found. The compounds are effective in treating capillary hemangiomas by reducing the growth and/or size of a capillary hemangioma when applied topically to the site of the capillary hemangioma on the skin of a human. Preferably, the capillary hemangioma is significantly reduced in size, preferably no longer visible, and more preferably, eliminated.

Anti-capillary hemangioma active compounds include xylometazoline, epinephrine, nerepinephrine, phenylephrine, methoxamine. Additional anti-capillary hemangioma active compounds include guanabenz, guanfacine, alpha-methyl DOPA (methyldopamine), amphetamine, methylphenidate, lofexidine, moxonidine, dexmedetomidine, and mivazerol. The preferred alpha-2 adrenergic receptor agonists are brimonidine and oxymetazoline. Each of the anti-capillary hemangioma active compounds may be in the form of a pharmaceutically acceptable salt thereof, as well as combinations of such compounds and/or of such salts

Brimonidine is 5-bromo-6-(2-imidazolidinylideneamino) quinoxaline. Its structure is shown below.

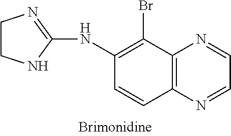 [SMILES: Brc1c(NC2=NCCN2)ccc2nccnc12]

The structure of oxymetazoline is shown below.

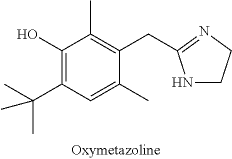 [SMILES: Cc1cc(C(C)(C)C)c(O)c(C)c1CC1=NCCN1]

Pharmaceutically acceptable salts thereof, as used herein, include those salts of the compounds of the invention that are safe and effective for topical use in mammals and that possess the desired biological activity. Pharmaceutically acceptable salts include, for example, acid addition salts of basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, aspartate, methanesulfonate, ethanesulfonate, benzensulfonate, p-toluenesulfonate and pamoate (i.e., 1,1′-methylene-bis-(2-hydroxy-3-naphthoate)) salts. Other pharmaceutically acceptable salts are described in Berge et al., 66 J. Pharm. Sci. 66, 1-19 (1977).

For example, brimonidine tartrate is the preferred salt of brimonidine. Oxymetazoline hydrochloride is the preferred salt of oxymetazoline.

The syntheses of the anti-capillary hemangioma active compounds described above are known in the art. For example, brimonidine or a pharmaceutically acceptable salt thereof and oxymetazoline or a pharmaceutically acceptable salt thereof can be synthesized by methods described in U.S. Pat. No. 7,439,241 and in Fuhrhop, et al. “Organic Synthesis: Concepts and Methods,” page 237-238 (2003).

### Pharmaceutically Acceptable Carriers

In one embodiment, the compounds of the invention are delivered to the affected area of the skin by a composition comprising a pharmaceutically acceptable topical carrier. As used herein, a pharmaceutically acceptable composition is any composition that can be applied to the skin surface for topical delivery of a pharmaceutical or medicament. Topical compositions of the invention may be prepared according to well-known methods in the art. For example, an anti-capillary hemangioma active compound of the invention may be combined with a topical carrier by methods provided in standard reference texts, such as, REMINGTON: THE SCIENCE AND PRACTICE OF PHARMACY 1577-1591, 1672-1673, 866-885 (Alfonso R. Gennaro ed. 19th ed. 1995); Ghosh, T. K.; et al. TRANSDERMAL AND TOPICAL DRUG DELIVERY SYSTEMS (1997).

The topical carriers useful for topical delivery of compounds of the invention can be any carrier known in the art for topically administering pharmaceuticals, for example, but not limited to, pharmaceutically acceptable solvents, such as a polyalcohol or water; emulsions (either oil-in-water or water-in-oil emulsions), such as creams, ointments, or lotions; micro emulsions; gels; liposomes; powders; aqueous solutions or suspensions.

### Emulsions and Gels as Topical Carriers

In a preferred embodiment, the topical carrier used to deliver a compound of the invention is an emulsion, e.g., a cream, lotion, or ointment; or a gel. Emulsions are suitable topical compositions for use in the invention. An emulsion is a dispersed system comprising at least two immiscible phases, one phase dispersed in the other as droplets, usually ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout an aqueous phase as droplets, the emulsion is termed an oil-in-water emulsion. Emulsions, such as creams, ointments and lotions, that can be used as topical carriers and their preparation are disclosed in REMINGTON: THE SCIENCE AND PRACTICE OF PHARMACY 282-291 (Alfonso R. Gennaro ed. 19th ed. 1995).

In one embodiment, the pharmaceutically acceptable carrier is a gel. Gels are semisolid systems that contain suspensions of inorganic particles, usually small inorganic particles, or organic molecules, usually large organic molecules, interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are known in the art, and may be two-phase or single-phase systems. Some examples of suitable gels are disclosed in REMINGTON: THE SCIENCE AND PRACTICE OF PHARMACY 1517-1518 (Alfonso R. Gennaro ed. 19^{th} ed. 1995). Other suitable gels for use with the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002).

Gelling agents, that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.).

“CARBOPOL®” is one of numerous cross-linked acrylic acid polymers that are given the general adopted name carbomer. “Carbomer” is the USP designation for various polymeric acids that are dispersible but insoluble in water. When the acid dispersion is neutralized with a base, a clear, stable gel is formed. The preferred carbomer is Carbomer 934P because it is physiologically inert and is not a primary irritant or sensitizer. Other carbomers include 910, 940, 941, and 1342.

Carbomers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration. Other suitable gelling agents include hydroxyethylcellulose, cellulose gum, MVE/MA decadiene crosspolymer, PVM/MA copolymer, or a combination thereof.

In a preferred embodiment, the minimum amount of gelling agent in the composition is about 0.5%, more preferably, about 0.75%, and most preferably about 1%. In another preferred embodiment, the maximum amount of gelling agent in the composition is about 2%, more preferably about 1.75%, and most preferably about 1.5%.

In another preferred embodiment, the topical carrier used to deliver a compound of the invention is an ointment. Ointments are oleaginous semisolids that contain little if any water. Preferably, the ointment is hydrocarbon based, such as a wax, petrolatum, or gelled mineral oil. Suitable ointments for use in the invention are well known in the art and are disclosed in REMINGTON: THE SCIENCE AND PRACTICE OF PHARMACY 1585-1591 (Alfonso R. Gennaro ed. 19th ed. 1995).

The pharmaceutical carrier may also be a cream. A cream is an emulsion, i.e., a dispersed system comprising at least two immiscible phases, one phase dispersed in the other as droplets usually ranging in diameter from 0.1 μm to 100 μm. An emulsifying agent is typically included to improve stability.

The pH of the pharmaceutical carrier is adjusted with, for example, a base such as sodium hydroxide or potassium hydroxide; or an amine base, such as trimethylamine. The minimum pH of the carrier is about 5, preferably 5.5, and most preferably 6.2 when the carrier is diluted by a factor of ten. The maximum pH of the carrier is about 8, preferably about 7.5, more preferably 7, and most preferably about 6.8 when the carrier is diluted by a factor of ten. Each minimum pH value can be combined with each maximum pH value to create various pH ranges. For example, the pH may be a minimum of 6.2 and a maximum of 7.5.

The pH values given above are those that occur if the composition is diluted with water by a factor of ten. It is not necessary to dilute the composition by a factor of ten in order to obtain a pH value. In practice, the composition may be diluted by any value that permits pH to be measured. For example, the composition may be diluted by a factor of about five to about twenty.

### Aqueous Topical Compositions of the Invention

In another embodiment, the topical carrier used in the topical compositions of the invention is an aqueous solution or suspension, preferably, an aqueous solution. Well-known solutions and suspensions are suitable topical carriers for use in the invention. Suitable aqueous topical compositions for use in the invention are disclosed in REMINGTON: THE SCIENCE AND PRACTICE OF PHARMACY 1563-1576 (Alfonso R. Gennaro ed. 19th ed. 1995). Other suitable aqueous topical carrier systems are disclosed in U.S. Pat. No. 5,424,078 (issued Jun. 13, 1995); U.S. Pat. No. 5,736,165 (issued Apr. 7, 1998); U.S. Pat. No. 6,194,415 (issued Feb. 27, 2001); U.S. Pat. No. 6,248,741 (issued Jun. 19, 2001); U.S. Pat. No. 6,465,464 (issued Oct. 15, 2002).

Tonicity-adjusting agents can be included in the aqueous topical compositions of the invention. Examples of suitable tonicity-adjusting agents include, but are not limited to, sodium chloride, potassium chloride, mannitol, dextrose, glycerin, and propylene glycol. The amount of the tonicity agent can vary widely depending on the composition's desired properties. In one embodiment, the tonicity-adjusting agent is present in the aqueous topical composition in an amount of from about 0.5 to about 0.9 weight percent of the composition.

The viscosity of aqueous solutions of the invention can be any convenient viscosity, and can be adjusted by adding viscosity adjusting agents, for example, but not limited to, polyvinyl alcohol, povidone, hydroxypropyl methyl cellulose, poloxamers, carboxymethyl cellulose, or hydroxyethyl cellulose. In one embodiment, the aqueous topical compositions of the invention have a viscosity in the range of from about 15 cps to about 25 cps.

In a preferred embodiment, the aqueous topical composition of the invention is an isotonic saline solution, optionally comprising a preservative, such as benzalkonium chloride or chlorine dioxide, a viscosity-adjusting agent, such as polyvinyl alcohol, and/or a buffer system such as sodium citrate and citric acid, or potassium acetate and acetic acid.

### Excipients

The topical compositions of the invention can further comprise pharmaceutically acceptable excipients such as those listed in REMINGTON: THE SCIENCE AND PRACTICE OF PHARMACY 866-885 (Alfonso R. Gennaro ed. 19th ed. 1995; Ghosh, T. K. et al., TRANSDERMAL AND TOPICAL DRUG DELIVERY SYSTEMS (1997)), including, but not limited to, protective agents, adsorbents, demulcents, emollients, preservatives, antioxidants, moisturizers, buffering agents, solubilizing agents, skin-penetration agents, and surfactants. Excipients are non-active and non-essential ingredients in the composition that do not materially affect the basic characteristics of the composition.

Suitable protective agents and adsorbents include, but are not limited to, dusting powders, zinc stearate, collodion, dimethicone, silicones, zinc carbonate, aloe vera gel and other aloe products, vitamin E oil, allatoin, glycerin, petrolatum, and zinc oxide.

Suitable demulcents include, but are not limited to, benzoin, hydroxypropyl cellulose, hydroxypropyl methylcellulose, and polyvinyl alcohol.

Suitable emollients include, but are not limited to, animal and vegetable fats and oils, myristyl alcohol, alum, and aluminum acetate.

Suitable preservatives include, but are not limited to, parabens, phenoxyethanol, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin.

Chlorine dioxide (ClO₂), preferably, stabilized chlorine dioxide, is a suitable preservative for use with topical compositions of the invention. The term “stabilized chlorine dioxide” is well known in the industry and by those skilled in the art. Stabilized chlorine dioxide includes one or more chlorine dioxide precursors such as one or more chlorine dioxide-containing complexes and/or one or more chlorite-containing components and/or one or more other entities capable of decomposing or being decomposed in an aqueous medium to form chlorine dioxide. U.S. Pat. No. 5,424,078 (issued Jun. 13, 1995) discloses a form of stabilized chlorine dioxide and a method for producing same, which can be used as a preservative for aqueous solutions and is useful in topical compositions of the invention. The manufacture or production of certain stabilized chlorine dioxide products is described in U.S. Pat. No. 3,278,447. A commercially available stabilized chlorine dioxide that can be utilized in the practice of the present invention is the proprietary stabilized chlorine dioxide of BioCide International, Inc. of Norman, Okla., sold under the trademark Purogene™ or Purite™ Other suitable stabilized chlorine dioxide products include that sold under the trademark DuraKlor by Rio Linda Chemical Company, Inc., and that sold under the trademark Antheium Dioxide by International Dioxide, Inc.

Suitable antioxidants include, but are not limited to, ascorbic acid and its esters, sodium bisulfite, butylated hydroxytoluene, butylated hydroxyanisole, tocopherols, and chelating agents like EDTA and citric acid.

Suitable moisturizers include, but are not limited to, glycerin, sorbitol, polyethylene glycols, urea, and propylene glycol.

Suitable buffering agents for use with the invention include, but are not limited to, acetate buffers, citrate buffers, phosphate buffers, lactic acid buffers, and borate buffers.

Suitable solubilizing agents include, but are not limited to, quaternary ammonium chlorides, cyclodextrins, benzyl benzoate, lecithin, and polysorbates.

Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methyl pyrrolidone.

### Additional Pharmaceutical Active Compounds

In one embodiment, the only anti-capillary hemangioma active compound in the composition is xylometazoline, epinephrine, nerepinephrine, phenylephrine, methoxamine, guanabenz, guanfacine, alpha-methyl dopamine, amphetamine, methylphenidate, lofexidine, moxonidine, dexmedetomidine, mivazerol, brimonidine, oxymetazoline, a pharmaceutically acceptable salt of any such compound, or any combination of such compounds or salts. In yet another embodiment, the only pharmaceutically active compound for treating capillary hemangiomas is brimonidine or a pharmaceutically acceptable salt thereof or oxymetazoline or a pharmaceutically acceptable salt thereof. In still another embodiment, the only two anti-capillary hemangioma active compounds in the composition are brimonidine or a pharmaceutically acceptable salt thereof and oxymetazoline or a pharmaceutically acceptable salt thereof.

In a further embodiment, the only active compound of any kind in the composition is xylometazoline, epinephrine, nerepinephrine, phenylephrine, methoxamine, guanabenz, guanfacine, alpha-methyl dopamine, amphetamine, methylphenidate, lofexidine, moxonidine, dexmedetomidine, mivazerol, brimonidine, oxymetazoline, a pharmaceutically acceptable salt thereof, or any combination of such compounds or salts. In yet another embodiment, the only active compound of any kind is brimonidine or a pharmaceutically acceptable salt thereof or oxymetazoline or a pharmaceutically acceptable salt thereof. In still another embodiment, the only two active compounds of any kind in the composition are brimonidine or a pharmaceutically acceptable salt thereof and oxymetazoline or a pharmaceutically acceptable salt thereof.

In another embodiment, one or more additional pharmaceutically active ingredients are included in the compositions of the invention, including a composition containing brimonidine or a pharmaceutically acceptable salt thereof, oxymetazoline or a pharmaceutically acceptable salt thereof, or any combinations of such compounds or salts. Additional active ingredients may include any pharmaceutically active ingredient. For example, the one or more additional pharmaceutically active ingredients may include, but are not limited to, antibacterial agents, anthelmintic agents, antioxidant agents, steroidal anti-inflammatory agents, non-steroidal anti-inflammatory agents, antiangiogenic agents, and derivatives of retinoic acid.

### Dosage

Dosages and dosing frequency of an effective amount of the compounds of the invention can be determined by trained medical professionals, typically during pre-clinical and clinical trials. The dosages and dosing frequency depend on numerous factors, such as the activity of the compounds of the invention, the characteristics of the particular topical composition, and the identity and severity of the capillary hemangiomas being treated.

In general, the active compounds described above are present in a composition of the invention in a minimum amount of about 0.01%, 0.05%, 0.1%, 0.15%, 0.2%, 0.25%, 0.3%, 0.35%, 0.4%, or 0.5% based upon the total weight of the composition. Generally, the active compounds described above are present in a composition of the invention in a maximum amount of about 5% 4%, 3%, 2%, 1%, 0.9%, 0.8%, 0.7%, or 0.6% based upon the total weight of the composition. For example, some dosages of brimonidine tartrate are 0.07%, 0.18%, and 0.5%.

### Topical Administration

The pharmaceutical compositions of the invention may be applied directly to the affected area of the skin in any manner known in the art. For example, a solution may be applied by cotton swab or may be sprayed. A suspension or an emulsion may be applied with a Q-tip® or an applicator stick, or by simply spreading a composition of the invention onto the affected area with one or more fingers. Preferably, the pharmaceutical compositions of the invention is applied only to skin, and is not administered to eyes.

Generally the amount of a topical composition of the invention applied to the affected skin area ranges from about 0.0001 g/cm² of skin surface area to about 0.01 g/cm², preferably, 0.001 g/cm² to about 0.003 g/cm² of skin surface area. Typically, one to four applications per day are recommended during the term of treatment.

### MISCELLANEOUS DEFINITIONS

It is to be understood that the present invention contemplates embodiments in which each minima is combined with maxima to create all feasible ranges. For example, either (1) brimonidine or a pharmaceutically acceptable salt thereof or (2) oxymetazoline or a pharmaceutically acceptable salt thereof may be present in a composition of the invention in an amount of from about 0.01 percent to about 5 percent based upon the total weight of the composition, preferably, from about 0.1 percent to about 1 percent based upon the total weight of the composition, or more preferably, from about 0.1 percent to about 0.5 percent based upon the total weight of the composition.

### EXAMPLES

### Example 1a

### Gel Composition

| Ingredient | Weight Percent |
|---|---|
| Brimonidine tartrate | 0.18% |
| Carbomer 934P | 1.25% |
| Methylparaben | 0.2% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

### Example 1b

### Gel Composition

| Ingredient | Weight Percent |
|---|---|
| Oxymetazoline hydrochloride | 0.2% |
| Carbomer 934P | 1.25% |
| Methylparaben | 0.2% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

### Example 1c

### Gel Composition

| Ingredient | Weight Percent |
|---|---|
| Brimonidine tartrate | 0.18% |
| Oxymetazoline hydrochloride | 0.2% |
| Carbomer 934P | 1.25% |
| Methylparaben | 0.2% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

### Example 2a

### Cream Composition

| Ingredient | Weight Percent |
|---|---|
| Brimonidine tartrate | 0.5% |
| Phenoxyethanol | 0.8% |
| Methylparaben | 0.2% |
| Propylparaben | 0.05% |
| Disodium EDTA | 0.01% |
| Butylated Hydroxytoluene | 0.05% |
| PEG-300 | 4.0% |
| PEG-6 Stearate (and) Glycol | 7.5% |
| Stearate (and) PEG-32 Stearate | |
| Cetostearyl alcohol | 4.0% |
| Caprylic capric triglycerides | 7.0% |
| Diisopropyl adipate | 7.0% |
| Oleyl alcohol | 7.0% |
| Lanolin USP | 2.0% |
| Ceteareth-6 (and) Stearyl | 2.0% |
| Alcohol | |
| Ceteareth-25 | 2.0% |
| Tartaric Acid | 0.001% |
| DI Water | 55.389% |
| TOTAL | 100% |

### Example 2b

### Cream Composition

| Ingredient | Weight Percent |
|---|---|
| Oxymetazoline hydrochloride | 0.5% |
| Phenoxyethanol | 0.8% |
| Methylparaben | 0.2% |
| Propylparaben | 0.05% |
| Disodium EDTA | 0.01% |
| Butylated Hydroxytoluene | 0.05% |
| PEG-300 | 4.0% |
| PEG-6 Stearate (and) Glycol | 7.5% |
| Stearate (and) PEG-32 Stearate | |
| Cetostearyl alcohol | 4.0% |
| Caprylic capric triglycerides | 7.0% |
| Diisopropyl adipate | 7.0% |
| Oleyl alcohol | 7.0% |
| Lanolin USP | 2.0% |
| Ceteareth-6 (and) Stearyl | 2.0% |
| Alcohol | |
| Ceteareth-25 | 2.0% |
| Tartaric Acid | 0.001% |
| DI Water | 55.389% |
| TOTAL | 100% |

### Example 2c

### Cream Composition

| Ingredient | Weight Percent |
|---|---|
| Brimonidine tartrate | 0.5% |
| Oxymetazoline hydrochloride | 0.5% |
| Phenoxyethanol | 0.8% |
| Methylparaben | 0.2% |
| Propylparaben | 0.05% |
| Disodium EDTA | 0.01% |
| Butylated Hydroxytoluene | 0.05% |
| PEG-300 | 4.0% |
| PEG-6 Stearate (and) Glycol | 7.5% |
| Stearate (and) PEG-32 Stearate | |
| Cetostearyl alcohol | 4.0% |
| Caprylic capric triglycerides | 7.0% |
| Diisopropyl adipate | 7.0% |
| Oleyl alcohol | 7.0% |
| Lanolin USP | 2.0% |
| Ceteareth-6 (and) Stearyl | 2.0% |
| Alcohol | |
| Ceteareth-25 | 2.0% |
| Tartaric Acid | 0.001% |
| DI Water | 55.389% |
| TOTAL | 100% |

### Example 3a

### Ointment Composition

| Ingredient | Weight Percent |
|---|---|
| Brimonidine tartrate | 5.0% |
| Cholesterol | 3.0% |
| Stearyl Alcohol | 3.0% |
| White Wax | 8.0% |
| White Petroleum | 76.0% |
| TOTAL | 100% |

### Example 3b

### Ointment Composition

| Ingredient | Weight Percent |
|---|---|
| Oxymetazoline hydrochloride | 5.0% |
| Cholesterol | 3.0% |
| Stearyl Alcohol | 3.0% |
| White Wax | 8.0% |
| White Petroleum | 76.0% |
| TOTAL | 100% |

### Example 3c

### Ointment Composition

| Ingredient | Weight Percent |
|---|---|
| Brimonidine tartrate | 5.0% |
| Oxymetazoline hydrochloride | 5.0% |
| Cholesterol | 3.0% |
| Stearyl Alcohol | 3.0% |
| White Wax | 8.0% |
| White Petroleum | 76.0% |
| TOTAL | 100% |

### Example 4a

### Aqueous Solution

An aqueous solution of the invention includes brimonidine tartrate (0.07 wt %); Purite® (0.005%) (stabilized chlorine dioxide) as a preservative; and the inactive ingredients: boric acid; calcium chloride; magnesium chloride; potassium chloride; purified water; sodium borate; sodium carboxymethylcellulose; sodium chloride; with hydrochloric acid and/or sodium hydroxide to adjust the pH to 5.6 to 6.6. The osmolality is in the range of 250-350 mOsmol/kg.

### Example 4b

### Aqueous Solution

An aqueous solution of the invention includes oxymetazoline hydrochloride (0.07 wt %); Purite® (0.005%) (stabilized chlorine dioxide) as a preservative; and the inactive ingredients: boric acid; calcium chloride; magnesium chloride; potassium chloride; purified water; sodium borate; sodium carboxymethylcellulose; sodium chloride; with hydrochloric acid and/or sodium hydroxide to adjust the pH to 5.6 to 6.6. The osmolality is in the range of 250-350 mOsmol/kg.

### Example 4c

### Aqueous Solution

An aqueous solution of the invention includes brimonidine tartrate (0.07 wt %); oxymetazoline hydrochloride (0.07 wt %); Purite® (0.005%) (stabilized chlorine dioxide) as a preservative; and the inactive ingredients: boric acid; calcium chloride; magnesium chloride; potassium chloride; purified water; sodium borate; sodium carboxymethylcellulose; sodium chloride; with hydrochloric acid and/or sodium hydroxide to adjust the pH to 5.6 to 6.6. The osmolality is in the range of 250-350 mOsmol/kg.

We claim:

1. A method for treating capillary hemangiomas in a human in need thereof, the method comprising topically administering to the site of capillary hemangiomas on the skin of the human a composition comprising an effective amount of one or more anti-capillary hemangioma active compounds selected from the group consisting of xylometazoline, epinephrine, nerepinephrine, phenylephrine, methoxamine, guanabenz, guanfacine, alpha-methyl dopamine, amphetamine, methylphenidate, lofexidine, moxonidine, dexmedetomidine, mivazerol, brimonidine, and oxymetazoline, pharmaceutically acceptable salts thereof, or any combinations of such compounds or salts.

2. The method according to claim 1, wherein the one or more active compounds or pharmaceutically acceptable salts thereof is selected from the group consisting of xylometazoline, epinephrine, nerepinephrine, phenylephrine, and methoxamine, or any combinations of such compounds or salts.

3. The method according to claim 1, wherein the one or more active compounds or pharmaceutically acceptable salts thereof is selected from the group consisting of guanabenz, guanfacine, alpha-methyl dopamine, amphetamine, methylphenidate, lofexidine, moxonidine, dexmedetomidine, and mivazerol, or any combinations of such compounds or salts.

4. The method according to claim 1, wherein the one or more active compounds or pharmaceutically acceptable salts thereof is selected from the group consisting of brimonidine, oxymetazoline, or pharmaceutically acceptable salts thereof, or a combination of any such compounds or salts.

5. The method according to claim 1, wherein the pharmaceutically acceptable salt is brimonidine tartrate.

6. The method according to claim 5, wherein the brimonidine tartrate is present in a minimum amount of about 0.01% and a maximum amount of about 5% based upon the total weight of the composition.

7. The method according to claim 1, wherein the pharmaceutically acceptable salt is oxymetazoline hydrochloride.

8. The method according to claim 7, wherein the oxymetazoline hydrochloriode is present in a minimum amount of about 0.01% and a maximum amount of about 5% based upon the total weight of the composition.

9. The method according to claim 1, wherein the only anti-capillary hemangioma active compound in the composition is xylometazoline, epinephrine, nerepinephrine, phenylephrine, methoxamine, guanabenz, guanfacine, alpha-methyl dopamine, amphetamine, methylphenidate, lofexidine, moxonidine, dexmedetomidine, mivazerol, brimonidine, oxymetazoline, a pharmaceutically acceptable salt thereof, or any combination of such compounds or salts.

10. The method according to claim 1, wherein the only pharmaceutically active compound for treating capillary hemangiomas are brimonidine or a pharmaceutically acceptable salt thereof or oxymetazoline or a pharmaceutically acceptable salt thereof; or a combination of brimonidine or a pharmaceutically acceptable salt thereof and oxymetazoline or a pharmaceutically acceptable salt thereof.

11. The method according to claim 1, wherein the only pharmaceutically active compound of any kind in the composition is brimonidine or a pharmaceutically acceptable salt thereof or oxymetazoline or a pharmaceutically acceptable salt thereof; or a combination of brimonidine or a pharmaceutically acceptable salt thereof or oxymetazoline or a pharmaceutically acceptable salt thereof.

12. The method of claim 1, wherein the composition further comprises one or more pharmaceutically active ingredient selected from the group consisting of antibacterial agents, anthelmintic agents, antioxidant agents, steroidal anti-inflammatory agents, non-steroidal anti-inflammatory agents, antiangiogenic agents, and derivatives of retinoic acid.

13. The method of claim 1, wherein the composition further comprises aloe or sunscreens, or combinations thereof.

14. The method of claim 1, wherein the composition further comprises a preservative.

15. The method of claim 1, further comprising systemically administering a corticosteroid.

16. The method of claim 1, further comprising intralesional injection of a corticosteroid.

17. The method of claim 1, further comprising radiation therapy of the capillary hemangiomas.

18. The method of claim 1, further comprising interferon therapy.

19. The method of claim 1, further comprising laser therapy.
